(19) United States
(12) Patent Application Publication
(10) Pub. No.: US 2017/0189323 A1
(43) Pub. Date: Jul. 6, 2017

(54) ALL-NATURAL SUNSCREEN

(71) Applicant: Ryan Charles Ballenas, Auburn, MA (US)

(72) Inventor: Ryan Charles Ballenas, Auburn, MA (US)

(21) Appl. No.: 15/083,464

(22) Filed: Mar. 29, 2016

Related U.S. Application Data
(60) Provisional application No. 62/272,971, filed on Dec. 30, 2015.

(51) Int. Cl. A61K 8/92; A61Q 17/04; A61K 8/27

(52) U.S. Cl. CPC A61K 8/927; A61K 8/27; A61Q 17/04; A61K 8/922; A61K 2800/5922

(57) ABSTRACT

A sunscreen composition composed of all-natural ingredients comprising a wax, a natural oil and zinc oxide.

### CROSS-REFERENCE TO RELATED APPLICATION

This application claims the benefit of priority of U.S. provisional application No. 62/272,971, filed Dec. 30, 2015, the contents of which are herein incorporated by reference.

### BACKGROUND OF THE INVENTION

At present, there are no sunscreen compositions available composed entirely of all natural substances. Most sunscreens are made with chemicals and other non-natural ingredients that may be non-eco-friendly and harmful to the environment and to an individual's health.

It is an object of the invention to provide a sunscreen composition containing all-natural ingredients.

### SUMMARY OF THE INVENTION

The above and other objects are realized by the present invention, one embodiment of which relates to an all-natural sunscreen composition comprising coconut oil, zinc oxide, beeswax, hempseed oil, red raspberry seed oil, carrot seed oil, shea butter, vitamin E oil, neem oil, rosehip oil, safflower oil, and babassu oil.

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a sunscreen composition composed of all-natural ingredients.

More particularly, a further embodiment of the invention relates to an all-natural sunscreen comprising at least one natural oil, at least one natural wax, and zinc oxide.

A still further embodiment of the invention is an all-natural sunscreen comprising:
1. coconut oil,
2. zinc oxide,
3. beeswax,
4. hempseed oil,
5. neem oil.
6. Rosehip oil,
7. Safflower oil, and
8. Babassu oil.

A more preferred embodiment of the invention concerns an all-natural sunscreen composition as described immediately above additionally containing:
9. red raspberry seed oil,
10. carrot seed oil,
11. Shea butter, and
12. vitamin E oil.

A still more preferred embodiment of the invention provides an all-natural sunscreen composition as described immediately above additionally containing:
13. almond oil,
14. grape-seed oil,
15. jojoba oil,
16. sunflower oil,
17. Sea buckthorn oil,
18. marula oil, and
19. Argan oil.

It will be understood by those skilled in the art that other natural oils may be added to or substituted for some of the oils listed above in the composition of the invention, such as, for example:
20. sesame oil,
21. avocado oil,
22. sweet almond oil,
23. apricot kernel oil,
24. kukui nut oil (candlenut oil),
25. wheat germ oil, and
26. cod-liver oil.

Additionally, any suitable natural wax may be utilized as a component of the all-natural sunscreen composition of the invention in addition to beeswax. Exemplary of such waxes are:
A. Microcrystalline Wax,
B. Ozokerite Wax,
C. Lanolin Alcohol,
D. Carnauba Wax, and
E. Candelilla Wax.
F. Japan Wax
G. Ceresine Wax
H. Laurel Wax
I. Tea Wax
J. Vegerite Wax
K. Bayberry Wax
L. The Natural Butters [avocado, sunflower, olive, etc.]

The wax functions as a bonding agent for the remaining ingredients in the composition.

The all-natural sunscreen composition of the invention also functions as skin moisturizing lotion, and insect repellent due to the insect repellent properties of neem oil.

The composition of the invention may be prepared according to the following general procedure: The natural was component is first carefully melted in a double boiler type system. The remaining ingredients are then added to the melted wax and the resulting mixture agitated until a homogenous composition is obtained. The mixture is allowed to cool until it reaches a consistency that enables its ready application to the skin of a user

### EXAMPLE

Following the above procedure, from about ¼ to about 1 cup of beeswax is placed in the inner container of a double boiler with water in the outer container. lo The water is brought to a slow boil for a time sufficient to completely melt the beeswax.

The following ingredients are then added to the melted wax:
1. ¼-½ cup hemp seed oil
2. ¾ cup coconut oil
3. 2-5 tablespoons zinc oxide (non-nano)
4. ¼-½ cup neem oil
5. 1-4 teaspoons rosehip oil
6. 1-4 tablespoons safflower oil
7. 1-4 tablespoons babassu oil
8. 1-4 teaspoons red raspberry seed oil
9. 1-4 teaspoons carrot seed oil
10.1 teaspoon vitamin E oil
11.2 tablespoons Shea Butter
12.1-4 teaspoons jojoba oil
13. 1-4 tablespoons grapeseed oil
14. 1-4 tablespoons sunflower oil
15. 1-4 teaspoons sea buckthorn oil
16.1-4 teaspoons marula oil
17. 1-4 teaspoons argan oil
18. ½ cup almond oil

The mixture is stirred until the composition attains homogeneity. It is then set aside at room temperature to cool. The resulting mixture has the consistency of a lotion which may be readily applied to the skin.

Those skilled in the art will appreciate that the amounts of the above ingredients may be changed in order that the consistency of the final composition reaches the desired level. For example, the use of more natural wax results in a final composition having a more solid paste-like consistency, whereas lessening the amount of natural wax reduces the consistency to that of an oil or lotion

It should be understood, of course, that the foregoing relates to exemplary embodiments of the invention and that modifications may be made without departing from the spirit and scope of the invention as set forth in the following claims.

What is claimed is:

1. A sunscreen composition composed of all-natural ingredients.

2. An all-natural sunscreen composition comprising at least one natural oil, at least one natural wax, and zinc oxide.

3. An all-natural sunscreen composition comprising coconut oil, zinc oxide, beeswax, hempseed oil, neem oil, rosehip oil, safflower oil, and babassu oil.

4. The all-natural sunscreen composition of claim 3, additionally containing red raspberry seed oil, carrot seed oil, Shea Butter, and vitamin E oil.

5. The all-natural sunscreen composition of claim 4, additionally containing almond oil, grape-seed oil, jojoba oil, sunflower oil, sea buckthorn oil, marula oil, and argan oil.

6. The all-natural sunscreen composition of claim 2, wherein said at least one natural oil is selected from the group consisting of sesame oil, safflower oil, avocado oil, sweet almond oil, apricot kernel oil, kukui nut oil (candlenut oil), wheat germ oil, and cod-liver oil.

7. The all-natural sunscreen composition of claim 2, wherein said at least one natural wax may comprise Microcrystalline Wax, Ozokerite Wax, Lanolin Alcohol, Carnauba Wax, or Candelilla Wax.
